(19) United States
(12) Patent Application Publication
(10) Pub. No.: US 2009/0306115 A1
(43) Pub. Date: Dec. 10, 2009

(54) Phenyl ethyne compounds

(76) Inventors: Nicholas D. Cosford, San Diego, CA (US); Thomas J. Seiders, San Diego, CA (US)

Correspondence Address: MERCK AND CO., INC, P O BOX 2000, RAHWAY, NJ, 07065-0907, US

(21) Appl. No.: 11/918,993

(22) PCT Filed: Apr. 18, 2006
(86) PCT No.: PCT/US2006/014517

§ 371 Date: Oct. 18, 2007

Related U.S. Application Data
(60) Provisional application No. 60/674,234, filed on Apr. 22, 2005.

(51) Int. Cl. A61K 31/506; C07D 211/76; C07D 239/08; A61K 31/4402; A61P 25/00

(52) U.S. Cl. 514/275; 546/303; 544/330; 514/345

(57) ABSTRACT

In accordance with the present invention, there is provided a novel class of heterocyclic compounds. Compounds of the invention contain a substituted or unsubstituted six membered heterocyclic ring that includes at least two nitrogen atoms. The ring additionally includes four carbon atoms. The heterocyclic ring has at least one substituent located at a ring position adjacent to a ring nitrogen atom. This mandatory substituent of the ring includes a moiety (B), linked to the heterocyclic ring via a carbon-carbon triple bond. The mandatory substituent is positioned adjacent to the ring nitrogen atom. Invention compounds are capable of a wide variety of uses. For example heterocyclic compounds can act to modulate physiological processes by functioning as agonists and antagonists of receptors in the nervous system. Invention compounds may also act as insecticides, and as fungicides. Pharmaceutical compositions containing invention compounds also have wide utility.

### BRIEF DESCRIPTION OF THE INVENTION

In accordance with the present invention, there is provided a novel class of compounds containing a substituted or unsubstituted phenyl ring “A” having at least one substituent which is a phenyl or heterocyclic moiety “B” linked to the phenyl A ring via an akynyl moiety.

The inventive compounds are useful for a wide variety of applications. For example the compounds can act to modulate physiological processes by functioning as agonists and antagonists of receptors in the nervous system. Inventive compounds may also act as insecticides, and as fungicides. Pharmaceutical compositions containing invention compounds also have wide utility.

### DETAILED DESCRIPTION OF THE INVENTION

In accordance with the present invention, there are provided compounds having the structure

A-L-B

wherein:
A is phenyl, unsubstituted or substituted with one or more substituent independently selected from:

(a) halogen,

(b) substituted or unsubstituted hydrocarbyl,

(c) substituted or unsubstituted aryl,

(d) substituted or unsubstituted heterocycle,

(e) mercapto,

(f) nitro,

(g) carboxyl,

(h) carbamate,

(i) carboxamide,

(j) hydroxy,

(k) ester,

(l) cyano,

(m) amine,

(n) amide,

(o) amidine,

(p) amido,

(q) sulfonyl or

(r) sulfonamide;

L is substituted or unsubstituted alkynylene; and
B is aryl or heterocycle unsubstituted or substituted with one or more substituent independently selected from:

(a) halogen,

(b) substituted or unsubstituted hydrocarbyl,

(c) substituted or unsubstituted aryl,

(d) substituted or unsubstituted heterocycle,

(e) mercapto,

(f) nitro,

(g) —O-heterocycle,

(h) —O-aryl

(i) carboxyl,

(j) carbamate,

(k) carboxamide,

(l) hydroxy,

(m) ester,

(n) cyano,

(o) amine,

(p) amide,

(q) amidine,

(r) amido,

(s) sulfonyl or

(t) sulfonamide;

and enantiomers, diastereomeric isomers or mixtures of any two or more thereof, or pharmaceutically acceptable salts thereof.

“Aryl” refers to mononuclear and polynuclear aromatic radicals having in the range of 6 up to 14 carbon atoms, and “substituted aryl” refers to aryl radicals further bearing one or more substituents as set forth above, for example, alkylaryl moieties.

“Heterocycle” refers to ring-containing radicals having one or more heteroatoms (e.g., N, O, S) as part of the ring structure, and having in the range of 3 up to 20 atoms in the ring. Heterocyclic moieties may be saturated or unsaturated when optionally containing one or more double bonds, and may contain more than one ring. Heterocyclic moieties include, for example, monocyclic moieties such as imidazolyl moieties, pyridinyl moieties, pyrimidinyl moieties, isothiazolyl moieties, isoxazolyl moieties, moieties, and the like, bicyclic heterocyclic moieties such as azabicycloalkanyl moieties, and oxabicycloalkyl moieties, and other non-aromatic and aromatic mon- and bicyclic heterocycles. The term “substituted heterocycle” refers to heterocycles further bearing one or more substituents as set forth above.

“Hydrocarbyl” refers to straight or branched chain univalent and bivalent radicals derived from saturated or unsaturated moieties containing only carbon and hydrogen atoms, and having in the range of about 1 up to 12 carbon atoms. Exemplary hydrocarbyl moieties include alkyl moieties, alkenyl moieties, dialkenyl moieties, trialkenyl moieties, alkynyl moieties, alkadiynal moieties, alkatriynal moieties, alkenyne moieties, alkadienyne moieties, alkenediyne moieties, and the like. The term “substituted hydrocarbyl” refers to hydrocarbyl moieties further bearing substituents as set forth below.

“Alkyl” refers to straight or branched chain alkyl radicals having in the range of about 1 up to 12 carbon atoms; “substituted alkyl” refers to alkyl radicals further bearing one or more substituents such as hydroxy, alkoxy, mercapto, aryl, heterocycle, halogen, trifluoromethyl, pentafluoroethyl, cyano, cyanomethyl, nitro, amino, amide, amidine, amido, carboxyl, carboxamide, carbamate, ester, sulfonyl, sulfonamide, and the like.

“Alkenyl” refers to straight or branched chain hydrocarbyl radicals having at least one carbon-carbon double bond, and having in the range of about 2 up to 12 carbon atoms (with radicals having in the range of about 2 up to 6 carbon atoms presently preferred), and “substituted alkenyl” refers to alkenyl radicals further bearing one or more substituents as set forth above. “Alkenylene” refers to straight or branched chain divalent alkenyl moieties having at least one carbon-carbon double bond, and having in the range of about 2 up to 12 carbon atoms (with divalent alkenyl moieties having in the range of about 2 up to 6 carbon atoms presently preferred), and “substituted lower alkenylene” refers to divalent alkenyl radicals further bearing one or more substituents as set forth above;

“Alkynyl” refers to straight or branched chain hydrocarbyl radicals having at least one carbon-carbon triple bond, and having in the range of about 2 up to 12 carbon atoms (with radicals having in the range of about 2 up to 6 carbon atoms presently being preferred), and “substituted alkynyl” refers to alkynyl radicals further bearing one or more substituents as set forth above. “Alkynylene” refers to straight or branched chain divalent alkynyl moieties having at least one carbon-carbon triple bond, and having in the range of about 2 up to 12 carbon atoms (with divalent alkynyl moieties having two carbon atoms presently being preferred), and “substituted alkynylene” refers to divalent alkynyl radicals further bearing one or more substituents as set forth above.

“Cyclohydrocarbyl” refers to cyclic (i.e., ring-containing) univalent radicals derived from saturated or unsaturated moieties containing only carbon and hydrogen atoms, and having in the range of about 3 up to 20 carbon atoms. Exemplary cyclohydrocarbyl moieties include cycloalkyl moieties, cycloalkenyl moieties, cycloalkadienyl moieties, cycloalkatrienyl moieties, cycloalkynyl moieties, cycloalkadiynyl moieties, spiro hydrocarbon moieties wherein two rings are joined by a single atom which is the only common member of the two rings (e.g., spiro[3.4]octanyl, and the like), bicyclic hydrocarbon moieties wherein two rings are joined and have two atoms in common (e.g., bicyclo[3.2.1]octane, bicyclo[2.2.1]hept-2-ene, and the like), and the like. The term “substituted cyclohydrocarbyl” refers to cyclohydrocarbyl moieties further bearing one or more substituents as set forth above;

“Cycloalkyl” refers to ring-containing radicals containing in the range of about 3 up to 20 carbon atoms, and “substituted cycloalkyl” refers to cycloalkyl radicals further bearing one or more substituents as set forth above;

“Cycloalkenyl” refers to ring-containing alkenyl radicals having at least one carbon-carbon double bond in the ring, and having in the range of about 3 up to 20 carbon atoms, and “substituted cycloalkenyl” refers to cyclic alkenyl radicals further bearing one or more substituents as set forth above.

“Azo” refers to the bivalent moiety —N═N—, wherein each bond is attached to a different carbon atom.

“Halogen” refers to fluoride, chloride, bromide or iodide radicals.

“Substituted,” including the use of “substituted” in reference to substituents of A and B, refers to the substituents recited above in connection with A and B. Thus, a substituent on A or B may itself be substituted with additional substituents selected from the A and B substituents. For instance, the substituent “substituted hydrocarbyl” may refer to a hydrocarbyl, such as methyl, that is further substituted with one or more of substituents (a) through (t), such as cyano. In this example, the resulting substituent would be —CH₂—CN. Similarly, the linker “L” may be further substituted with one or more of substituents (a) through (t).

Further, in accordance with the present invention, L is a linking moiety which links moieties A and B. L is selected from substituted or unsubstituted alkynylene moieties. Presently preferred compounds of the invention are those wherein L is an unsubstituted alkynylene moiety containing two carbon atoms, i.e., ethynyl.

Further, in accordance with the present invention, A is a moiety linked through bridging moiety L to moiety B. Radicals contemplated for use in the invention are those wherein A is substituted or unsubstituted phenyl. Preferred compounds of the invention are those wherein A is phenyl unsubstituted or substituted with one or more substituent independently selected from amino, alkyl, cyano, halogen, alkyl-cyano, alkyl-hydroxy, hydroxyl, alkoxy, and mercapto.

Further, in accordance with the present invention, B is a moiety linked through bridging moiety L to moiety A. Radicals contemplated for use in the invention are those wherein B is substituted or unsubstituted aryl or heterocycle. Further, preferred compounds of the invention are those wherein B is a substituted or unsubstituted aryl or heterocycle. Exemplary moieties include phenyl and pyrimidinyl. Especially preferred compounds are those wherein B is phenyl substituted with cyano and fluoro, for instance wherein B is substituted at the 3 position with cyano and the 5 position with fluoro, or where B is substituted at the 3 position with —O-pyridyl and the 5 position with fluoro. Additional especially preferred compounds are those wherein B is pyrimidinyl substituted with a heterocycle, in particular piperidinyl.

Thus, preferred compounds are those of the formula:

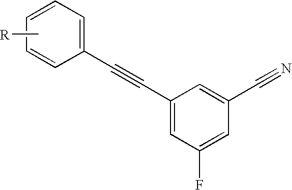

wherein R is a substituent independently selected from amino, alkyl, cyano, halogen, alkyl-cyano, alkyl-hydroxy, hydroxyl, alkoxy, and mercapto.

Additional preferred compound are those of the formula:

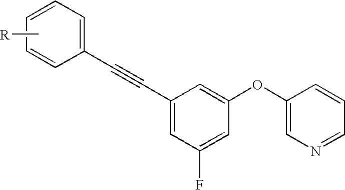

wherein R is a substituent independently selected from amino, alkyl, cyano, halogen, alkyl-cyano, alkyl-hydroxy, hydroxyl, alkoxy, and mercapto.

Still further preferred compound are those of the formula:

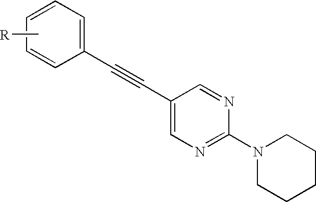

wherein R is a substituent independently selected from amino, alkyl, cyano, halogen, alkyl-cyano, alkyl-hydroxy, hydroxyl, alkoxy, and mercapto.

Those of skill in the art recognize that invention compounds may contain one or more chiral centers, and thus can exist as racemic mixtures. For many applications, it is preferred to carry out stereoselective syntheses and/or to subject the reaction product to appropriate purification steps so as to produce substantially optically pure materials. Suitable stereoselective synthetic procedures for producing optically pure materials are well known in the art, as are procedures for purifying racemic mixtures into optically pure fractions. Those of skill in the art will further recognize that invention compounds may exist in polymorphic forms wherein a compound is capable of crystallizing in different forms. Suitable methods for identifying and separating polymorphisms are known in the art.

In accordance with another embodiment of the present invention, there are provided pharmaceutical compositions comprising heterocyclic compounds as described above, in combination with pharmaceutically acceptable carriers. Optionally, invention compounds can be converted into non-toxic acid addition salts, depending on the substituents thereon. Thus, the above-described compounds (optionally in combination with pharmaceutically acceptable carriers) can be used in the manufacture of medicaments useful for the treatment of a variety of indications.

Pharmaceutically acceptable carriers contemplated for use in the practice of the present invention include carriers suitable for intravenous, subcutaneous, transcutaneous, intramuscular, intracutaneous, intrathecal, inhalation, intracranial, epidural, vaginal, oral, sublingual, rectal, and the like administration. Administration in the form of creams, lotions, tablets, dispersible powders, granules, syrups, elixirs, sterile aqueous or non-aqueous solutions, suspensions or emulsions, patches, and the like, is contemplated.

For the preparation of oral liquids, suitable carriers include emulsions, solutions, suspensions, syrups, and the like, optionally containing additives such as wetting agents, emulsifying and suspending agents, sweetening, flavoring and perfuming agents, and the like.

For the preparation of fluids for parental administration, suitable carriers include sterile aqueous or non-aqueous solutions, suspensions, or emulsions. Examples of non-aqueous solvents or vehicles are propylene glycol, polyethylene glycol, vegetable oils, such as olive oil and corn oil, gelatin, and injectable organic esters such as ethyl oleate. Such dosage forms may also contain adjuvants such as preserving, wetting, emulsifying, and dispersing agents. They may be sterilized, for example, by filtration through a bacteria-retaining filter, by incorporating sterilizing agents into the compositions, by irradiating the compositions, or by heating the compositions. They can also be manufactured in the form of sterile water, or some other sterile injectable medium immediately before use.

Invention compounds can optionally be converted into non-toxic acid addition salts. Such salts are generally prepared by reacting the compounds of this invention with a suitable organic or inorganic acid. Representative salts include hydrochloride, hydrobromide, sulfate, bisulfate, methanesulfonate, acetate, oxalate, adipate, alginate, aspartate, valerate, oleate, laurate, borate, benzoate, lactate, phosphate, toluenesulfonate (tosylate), citrate, malate, maleate, fumarate, succinate, tartrate, napsylate, methanesulfonate, 2-naphthalenesulfonate, nicotinate, benzenesulfonate, butyrate, camphorate, camphorsulfonate, cyclopentanepropionate, digluconate, dodecylsulfate, glucoheptanoate, glycerophosphate, heptanoate, hexanoate, undecanoate, 2-hydroxyethanesulfonate, ethanesulfonate, and the like. Salts can also be formed with inorganic acids such as sulfate, bisulfate, hemisulfate, hydrochloride, chlorate, perchlorate, hydrobromide, hydroiodide, and the like. Examples of a base salt include ammonium salts; alkali metal salts such as sodium salts, potassium salts, and the like; alkaline earth metal salts such as calcium salts, magnesium salts, and the like; salts with organic bases such as dicyclohexylamine salts, N-methyl-D-glucamine, phenylethylamine, and the like; and salts with amino acids such as arginine, lysine, and the like. Such salts can readily be prepared employing methods well known in the art.

In accordance with another embodiment of the present invention, there are provided methods for the preparation of heterocyclic compounds as described above. For example, many of the heterocyclic compounds described above can be prepared using synthetic chemistry techniques well known in the art (see Comprehensive Heterocyclic Chemistry, Katritzky, A. R. and Rees, C. W. eds., Pergamon Press, Oxford, 1984, and WO01/16121).

The following examples are intended to illustrate but not to limit the invention in any manner, shape, or form, either explicitly or implicitly. While they are typical of those that might be used, other procedures, methodologies, or techniques known to those skill in the art may alternatively be used.

### Intermediate 1

### 3-bromo-5-fluorobenzonitrile

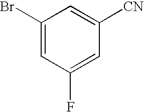

3,5-dibromofluorobenzene (47 g, 185 mmol), copper cyanide (16.5 g, 185 mmol), and DMF (300 mL) were heated to 140° C. for 18 hours. The reaction was cooled to room temperature, extracted with EtOAc/hexanes (1:1) and washed with aqueous ammonium hydroxide three times. The crude material was purified on silica with 10% EtOAc/hexanes as the eluent to yield a white solid.

### Intermediate 2

### 3-ethynyl-5-fluorobenzonitrile

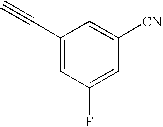

3-bromo-5-cyanofluorobenezene (7 g, 35 mmol), TMS acetylene (5.1 g, 53 mmol), Palladium tetrakis(triphenylphosphine) (0.4 g, 0.35 mmol), copper (I) iodide (0.07 g, 0.35 mmol), and triethylamine (100 mL) were combined and heated to 40° C. for 3 hours. The solution was filtered, solvent evaporated and the crude material purified on silica (20% EtOAc/hexanes) to yield a white solid. The solid material was dissolved in THF (50 mL) and 1 equivalent of TBAF was added and the solution was stirred for 1 hour at room temperature. Methylene chloride was added and the organic layer was washed 3 times with water and evaporated to yield a colorless oil.

### Example 1

### 3-[(3-cyanophenyl)ethynyl]-5-fluorobenzonitrile

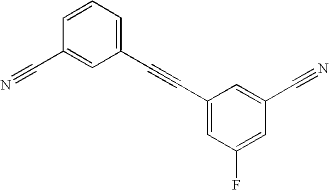

3-alkynyl-5-bromofluorobenzene (0.1 g, 0.7 mmol), 3-iodobenzonitrile (0.2 g, 1 mmol), Palladium tetrakis(triphenylphosphine) (0.02, 0.02 mol), copper (I) iodide (0.004, 0.02 mol), and triethylamine (1 mL), and DMF (2.5 mL) were combined and heated to 70° C. for 4 hours. The crude solution was filtered and purified directly on RPHPLC to yield an off white solid. ¹H NMR 7.85 (m, 1H), 7.78 (m, 1H), 7.71 (m, 1H), 7.65 (m, 1H), 7.54 (m, 1H), 7.50 (m, 1H), 7.39 (m, 1H).

Using methods and procedures similar to those described in Intermediates 1 and 2, and in Example 1 (above), the compounds described in Examples 2 through 16 were made.

### Example 2

### 3-fluoro-5-(phenylethynyl)benzonitrile

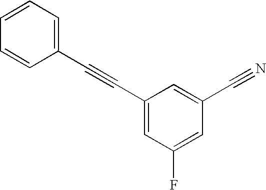

¹H NMR 7.64 (s, 1H), 7.55 (m, 2H), 7.48 (m, 1H), 7.44 (m, 3H), 7.34 (m, 1H).

### Example 3

### 3-[(3-aminophenyl)ethynyl]-5-fluorobenzonitrile

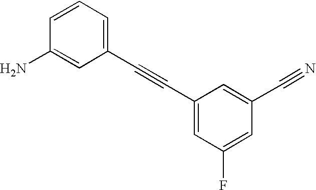

¹H NMR 7.61 (s, 1H), 7.44 (m, 1H), 7.33 (m, 1H), 7.18 (t, 1H), 6.96 (m, 1H), 6.86 (m, 1H), 6.73 (m, 1H).

### Example 4

### 3-fluoro-5-[(3-methylphenyl)ethynyl]benzonitrile

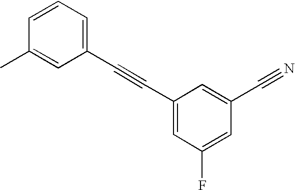

¹H NMR 7.61 (s, 1H), 7.46 (m, 1H), 7.38-7.31 (m, 4H), 7.25 (m, 1H).

### Example 5

### 3-fluoro-5-[(3-fluorophenyl)ethynyl]benzonitrile

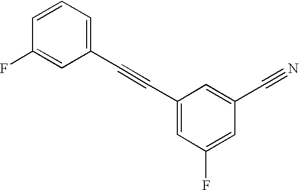

¹H NMR 7.64 (s, 1H), 7.46 (m, 1H), 7.38-7.33 (m, 3H), 7.25 (m, 1H), 7.15 (m, 1H).

### Example 6

### 3-{[3-(cyanomethyl)phenyl]ethynyl}-5-fluorobenzonitrile

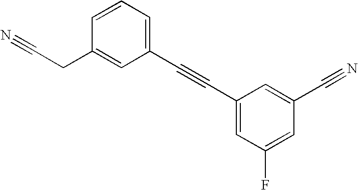

¹H NMR 7.64 (s, 1H), 7.55 (m, 2H), 7.47 (m, 1H), 7.43 (m, 1H), 7.38 (m, 2H), 3.80 (s, 2H).

### Example 7

### 3-fluoro-5-{[3-(hydroxymethyl)phenyl]ethynyl}benzonitrile

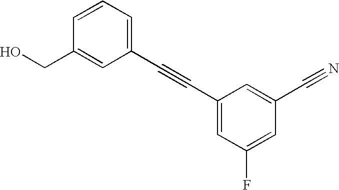

¹H NMR 7.60 (s, 1H), 7.57 (m, 1H), 7.47 (m, 2H), 7.41 (m, 2H), 7.33 (m, 1H), 4.73 (s, 2H).

### Example 8

### 3-[(3-chlorophenyl)ethynyl]-5-fluorobenzonitrile

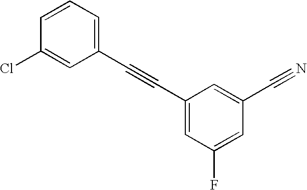

¹H NMR 7.85 (m, 1H), 7.78 (m, 1H), 7.71 (m, 1H), 7.65 (m, 1H), 7.54 (m, 1H), 7.50 (m, 1H), 7.39 (m, 1H).

### Example 9

### 3-[(3-bromophenyl)ethynyl]-5-fluorobenzonitrile

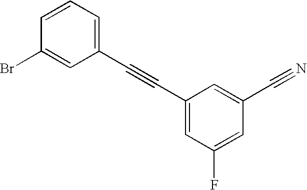

¹H NMR 7.71 (m, 1H), 7.63 (m, 1H), 7.56 (m, 1H), 7.47 (m, 2H), 7.38 (m, 1H), 7.29 (m, 1H).

### Example 10

### 3-fluoro-5-[(3-hydroxyphenyl)ethynyl]benzonitrile

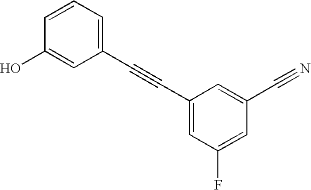

¹H NMR 7.62 (m, 1H), 7.47 (m, 1H), 7.34 (m, 1H), 7.28 (m, 1H), 7.12 (m, 1H), 7.02 (m, 1H), 6.92 (m, 1H).

### Example 11

### 3-fluoro-5-[(2-methylphenyl)ethynyl]benzonitrile

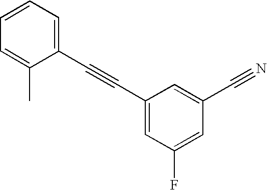

¹H NMR 7.62 (m, 1H), 7.52 (m, 1H), 7.47 (m, 1H), 7.31 (m, 2H), 7.29 (m, 1H), 7.21 (m, 1H), 2.53 (s, 3H).

### Example 12

### 3-fluoro-5-[(4-methylphenyl)ethynyl]benzonitrile

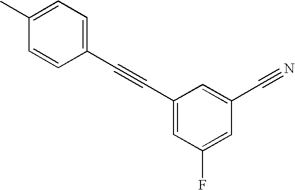

¹H N 7.62 (m, 1H), 7.45 (m, 3H), 7.31 (m, 1H), 7.21 (m, 2H), 2.40 (s, 3H).

### Example 13

### 3-[(2-cyanophenyl)ethynyl]-5-fluorobenzonitrile

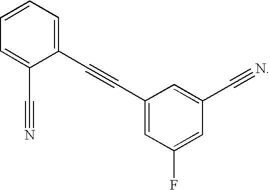

¹H NMR 7.75 (m, 1H), 7.71 (m, 1H), 7.66 (m, 2H), 7.58 (m, 1H), 7.52 (m, 1H), 7.40 (m, 1H).

### Example 14

### 3-[(4-cyanophenyl)ethynyl]-5-fluorobenzonitrile

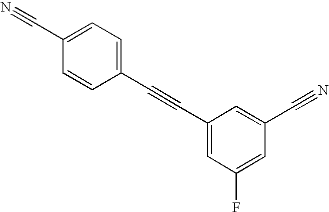

¹H NMR 7.70 (m, 2H), 7.63 (m, 3H), 7.50 (m, 1H), 7.40 (m, 1H).

### Example 15

### 3,3′-ethyne-1,2-diylbis(5-fluorobenzonitrile)

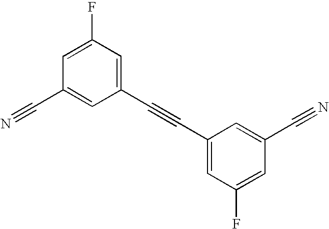

¹H NMR 7.65 (m, 2H), 7.48 (m, 2H), 7.41 (m, 2H).

### Intermediate 3

### 3-(3-bromo-5-fluorophenoxy)pyridine

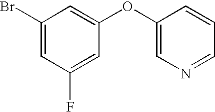

3,5-difluorobromofluoro (7 g, 28 mmol), 3-hydroxypyridine (5 g, 53 mmol), potassium carbonate (10 g) and DMF (300 mL) were heated to 140° C. for 18 hours. The reaction was cooled to room temperature, extracted with EtOAc/hexanes (1:1) and washed with water three times. The crude material was purified on silica with 20-40% EtOAc/hexanes as the eluent to yield a colorless oil.

### Intermediate 4

### 3-(3-ethynyl-5-fluorophenoxy)pyridine

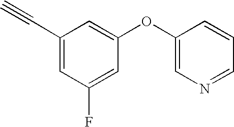

3-(3-bromo-5-fluorophenoxy)pyridine (7 g, 28 mmol), TMS acetylene (5.4 g, 55 mmol), Palladium tetrakis(triphenylphosphine) (1.3 g, 1.1 mmol), copper (I) iodide (0.21 g, 1.1 mmol), and triethylamine (100 mL) were combined and heated to 70° C. for 3 hours. The solution was filtered, solvent evaporated and the crude material purified on silica (25% EtOAc/hexanes) to yield a white solid. The solid material was dissolved in THF (50 mL) and 1 equivalent of TBAF was added and the solution was stirred for 1 hour at room temperature. Methylene chloride was added and the organic layer was washed 3 times with water and evaporated to yield a colorless oil.

### Example 16

### 3-{[3-fluoro-5-(pyridin-3-yloxy)phenyl]ethynyl}benzonitrile

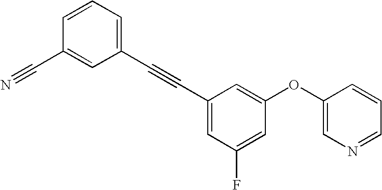

3-(3-ethynyl-5-fluorophenoxy)pyridine (0.1 g, 0.7 mmol), 3-iodobenzonitrile (0.2 g, 1 mmol), Palladium tetrakis(triphenylphosphine) (0.02, 0.02 mol), copper (I) iodide (0.004, 0.02 mol), and triethylamine (1 mL), and DMF (2.5 mL) were combined and heated to 70° C. for 4 hours. The crude solution was filtered and purified directly on RPHPLC to yield an off white solid. ¹H NMR 8.5 (b, 2H), 7.6-7.8 (m, 3H), 7.49 (m, 3H), 7.05 (m, 1H), 6.92 (s, 1H), 6.79 (m, 1H). M⁺+H, 315.0.

Using methods and procedures similar to those described in Intermediates 3 and 4, and in Example 16 (above), the compounds described in Examples 17 through 24 were made.

### Example 17

### 3-[3-fluoro-5-(phenylethynyl)phenoxy]pyridine

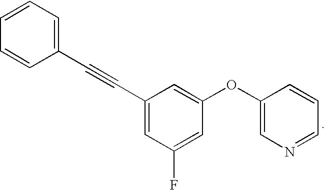

¹H NMR 8.5 (b, 2H), 7.55 (m, 2H), 7.45 (m, 2H), 7.38 (m, 3H), 7.05 (m, 1H), 6.97 (s, 1H), 6.76 (m, 1H). M⁺+H, 290.1.

### Example 18

### 3-{3-fluoro-5-[(3-methylphenyl)ethynyl]phenoxy}pyridine

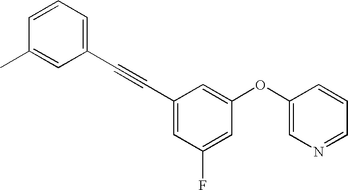

¹H NMR 8.5 (b, 2H), 7.15-7.48 (m, 6H), 7.05 (m, 1H), 6.92 (s, 1H), 6.74 (m, 1H), 2.35 (s, 3H). M⁺+H, 304.1.

### Example 19

### 3-{3-[(3-chlorophenyl)ethynyl]-5-fluorophenoxy}pyridine

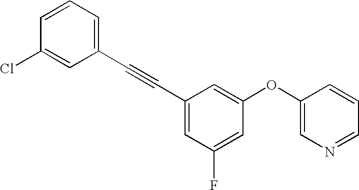

¹H NMR 8.5 (b, 2H), 7.2-7.5 (m, 6H), 7.03 (m, 1H), 6.92 (s, 1H), 6.75 (m, 1H). M⁺+H, 324.

### Example 20

### 3-fluoro-5-{[3-fluoro-5-(pyridin-3-yloxy)phenyl]ethynyl}benzonitrile

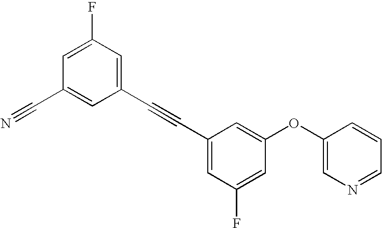

¹H NMR 7.3-7.6 (m, 4H), 7.05 (m, 1H), 6.92 (s, 1H), 6.79 (m, 1H). M⁺+H, 333.0.

### Example 21

### 3-{3-fluoro-5-[(2-methylphenyl)ethynyl]phenoxy}pyridine

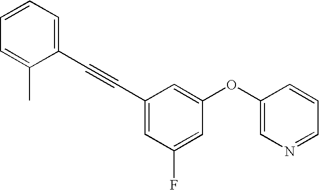

¹H NMR 8.5 (b, 2H), 7.5 (m, 3H), 7.2-7.3 (m, 3H), 7.05 (m, 1H), 6.92 (s, 1H), 6.74 (m, 1H), 2.5 (s, 3H). M⁺+H, 303.8.

### Example 22

### 3-{3-fluoro-5-[(4-methylphenyl)ethynyl]phenoxy}pyridine

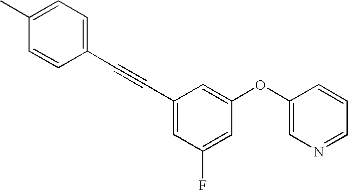

¹H NMR 8.5 (b, 2H), 7.4-7.5 (m, 4H), 7.3 (d, 2H), 7.05 (m, 1H), 6.92 (s, 1H), 6.74 (m, 1H), 2.4 (s, 3H). M⁺+H, 303.8.

### Example 23

### 2-{[3-fluoro-5-(pyridin-3-yloxy)phenyl]ethynyl}benzonitrile

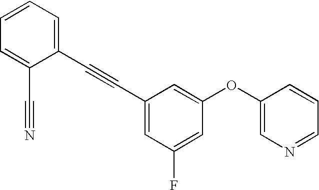

¹H NMR 7.4-7.8 (m, 4H), 7.2 (d, 2H), 7.17 (d, 1H), 7.06 (s, 1H), 6.8 (d, 1H). M⁺+H, 314.9.

### Example 24

### 4-{[3-fluoro-5-(pyridin-3-yloxy)phenyl]ethynyl}benzonitrile

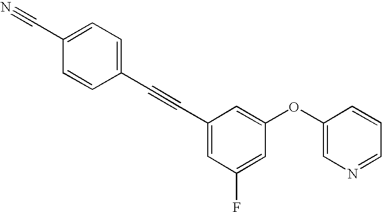

¹H NMR 7.4-7.9 (m, 7H), 7.08 (d, 1H), 6.97 (s, 1H), 6.8 (d, 1H). M⁺+H, 315.0.

### Intermediate 5

### 5-bromo-2-piperidin-1-ylpyrimidine

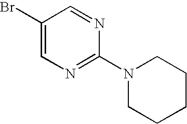

2-chloro-5-bromopyrimidine (3 g, 15.5 mmol), piperidine (5.3 g, 62 mmol), and DME (30 mL) were stirred at room temperature for 1 hour. The reaction was extracted with methylenechloride and washed with water three times and the solvent evaporated to yield and off white solid.

### Intermediate 6

### 5-ethynyl-2-piperidin-1-ylpyrimidine

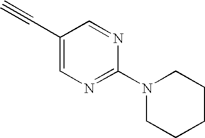

2-piperidyl-5-bromopyrimidine (3.5 g, 15 mmol), TMS acetylene (2.1 g, 29 mmol), Palladium tetrakis(triphenylphosphine) (0.3 g, 0.3 mmol), copper (I) iodide (0.05 g, 0.3 mmol), triethylamine (10 mL), and toluene (50 mL) were combined and heated to 100° C. for 12 hours. The solution was filtered, solvent evaporated and the crude material purified on silica (25% EtOAc/hexanes) to yield a white solid. The solid material was dissolved in THF (50 mL) and 1 equivalent of TBAF was added and the solution was stirred for 1 hour at room temperature. Methylene chloride was added and the organic layer was washed 3 times with water and evaporated to yield a white solid.

### Example 25

### 3-[(2-piperidin-1-ylpyrimidin-5-yl)ethynyl]benzonitrile

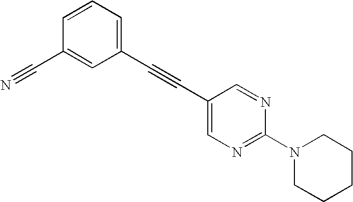

5-ethynyl-2-piperidin-1-ylpyrimidine (0.1 g, 0.5 mmol), 3-iodobenzonitrile (0.25 g, 1.1 mmol), Palladium tetrakis(triphenylphosphine) (0.02, 0.02 mol), copper (I) iodide (0.004, 0.02 mol), and triethylamine (1 mL), and DMF (2.5 mL) were combined and heated to 70° C. for 4 hours. The crude solution was filtered and purified directly on RPHPLC to yield an off white solid. ¹H NMR 8.4 (s, 2H), 7.4-7.8 (m, 4H), 3.9 (m, 7H), 2.5 (s, 3H), 1.6-1.7 (m, 6H). M⁺+H, 289.1.

Using methods and procedures similar to those described in Intermediates 5 and 6, and in Example 25 (above), the compounds described in Examples 26 through 33 were made.

### Example 26

### 5-(phenylethynyl)-2-piperidin-1-ylpyrimidine

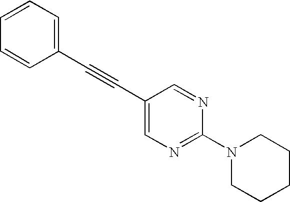

¹H NMR 8.4 (s, 2H), 7.5 (m, 2H), 7.35 (m, 3H), 3.9 (m, 4H), 1.6-1.7 (m, 6H). M⁺+H, 264.1.

### Example 27

### 5-[(3-methylphenyl)ethynyl]-2-piperidin-1-ylpyrimidine

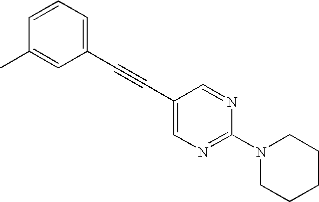

¹H NMR 8.4 (s, 2H), 7.1-7.4 (m, 4H), 3.9 (m, 4H), 1.6-1.7 (m, 6H). M⁺+H, 278.0.

### Example 28

### 5-[(3-chlorophenyl)ethynyl]-2-piperidin-1-ylpyrimidine

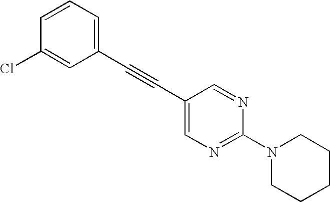

¹H NMR 8.4 (s, 2H), 7.3-7.5 (m, 4H), 3.9 (m, 4H), 1.6-1.7 (m, 6H). M⁺+H, 297.9.

### Example 29

### 5-[(3,5-dimethylphenyl)ethynyl]-2-piperidin-1-ylpyrimidine

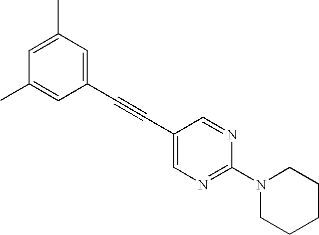

¹H NMR 8.4 (s, 2H), 7.2 (s, 1H), 6.9 (s, 1H), 3.9 (m, 4H), 1.6-1.7 (m, 6H).

### Example 30

### 5-[(3-methoxyphenyl)ethynyl]-2-piperidin-1-ylpyrimidine

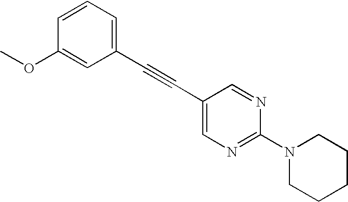

¹H NMR 8.4 (s, 2H), 7.3 (m, 1H), 7.1 (m, 2H), 6.9 (m, 1H), 3.9 (m, 7H), 1.6-1.7 (m, 6H). M⁺+H, 294.1.

### Example 31

### 5-{[3-methylthio)phenyl]ethynyl}-2-piperidin-1-ylpyrimidine

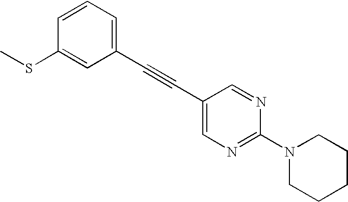

¹H NMR 8.4 (s, 2H), 7.2-7.4 (m, 4H), 3.9 (m, 7H), 2.5 (s, 3H), 1.6-1.7 (m, 6H). M⁺+H, 309.9

### Example 32

### {3-[(2-piperidin-1-ylpyrimidin-5-yl)ethynyl]benzyl}amine

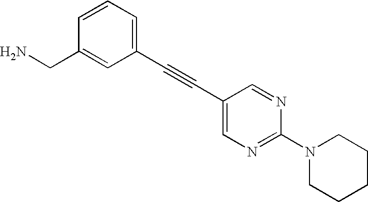

¹H NMR 8.4 (s, 2H), 7.2-7.5 (m, 4H), 4.0 (s, 2H), 3.9 (m, 7H), 2.5 (s, 3H), 1.6-1.7 (m, 6H). M⁺+H, 293.0.

### Example 33

### {3-[(2-piperidin-1-ylpyrimidin-5-yl)ethynyl]phenyl}acetonitrile

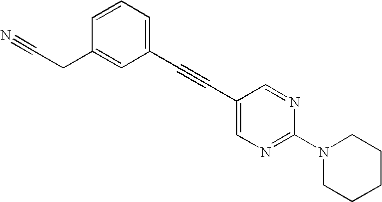

¹H NMR 8.4 (s, 2H), 7.2-7.5 (m, 4H), 3.9 (m, 7H), 3.77 (s, 21), 2.5 (s, 3H), 1.6-1.7 (m, 61). M⁺+H, 289.1.

While the invention has been described in detail with reference to certain preferred embodiments thereof, it will be understood that modifications and variations are within the spirit and scope of that which is described and claimed.

1-7. (canceled)

8. A compound of the formula:
A-L-B
wherein:
A is phenyl, which is unsubstituted or substituted with one or more substituents that are independently selected from the group consisting of:
(a) halogen,
(b) substituted or unsubstituted hydrocarbyl,
(c) substituted or unsubstituted aryl,
(d) substituted or unsubstituted heterocycle,
(e) mercapto,
(f) nitro,
(g) carboxyl,
(h) carbamate,
(i) carboxamide,
(j) hydroxy,
(k) ester,
(l) cyano,
(m) amine,
(n) amide,
(o) amidine,
(p) amido,
(q) sulfonyl or
(r) sulfonamide;
L is substituted or unsubstituted alkynylene; and
B is phenyl, pyridyl or pyrimidinyl, which is unsubstituted or substituted with one or more substituents that are independently selected from the group consisting of:
(a) halogen,
(b) substituted or unsubstituted hydrocarbyl,
(c) substituted or unsubstituted aryl,
(d) substituted or unsubstituted heterocycle,
(e) mercapto,
(f) nitro,
(g) —O-heterocycle,
(h) —O-aryl
(i) carboxyl,
(j) carbamate,
(k) carboxamide,
(l) hydroxy,
(m) ester,
(n) cyano,
(o) amine,
(p) amide,
(q) amidine,
(r) amido,
(s) sulfonyl, and
(t) sulfonamide;
or a pharmaceutically acceptable salt thereof.

9. The compound of claim 8 wherein B is phenyl.

10. The compound of claim 9 wherein B is phenyl which is substituted with cyano and fluoro.

11. The compound of claim 10 wherein B is phenyl which is substituted at the 3-position with cyano and substituted at the 5-position with fluoro.

12. The compound of claim 11 of the formula: 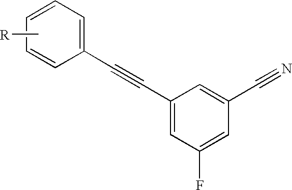
wherein:
R is selected from the group consisting of amino, alkyl, cyano, halogen, alkyl-cyano, alkyl-hydroxy, hydroxyl, alkoxy, and mercapto;
or a pharmaceutically acceptable salt thereof.

13. The compound of claim 10 wherein B is phenyl which is substituted at the 3-position with —O-pyridyl and substituted at the 5-position with fluoro.

14. The compound of claim 13 of the formula: 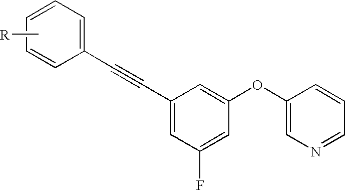
wherein:
R is selected from the group consisting of amino, alkyl, cyano, halogen, alkyl-cyano, alkyl-hydroxy, hydroxyl, alkoxy, and mercapto;
or a pharmaceutically acceptable salt thereof.

15. The compound of claim 8 wherein B is pyridyl.

16. The compound of claim 8 wherein B is pyrimidinyl.

17. The compound of claim 16 wherein B is pyrimidinyl which is substituted with piperidinyl.

18. The compound of claim 17 of the formula: 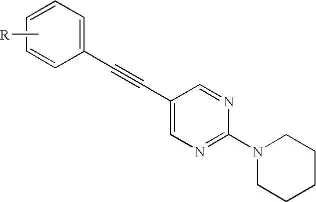
wherein:
R is selected from the group consisting of amino, alkyl, cyano, halogen, alkyl-cyano, alkyl-hydroxy, hydroxyl, alkoxy, and mercapto;
or a pharmaceutically acceptable salt thereof.

19. A compound which is selected from the group consisting of: 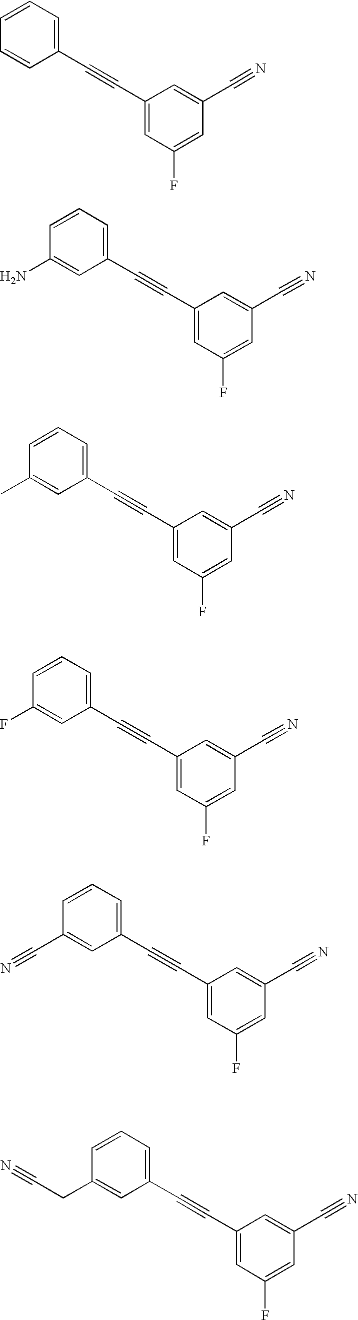 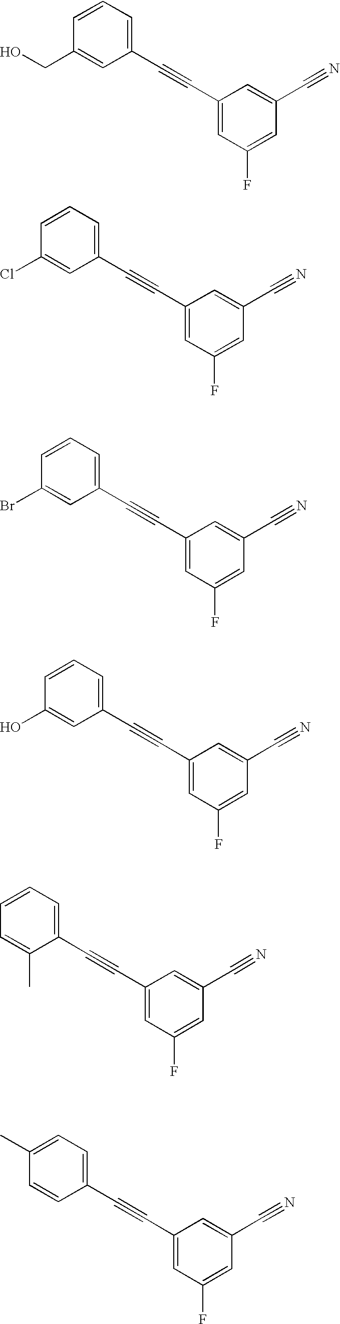 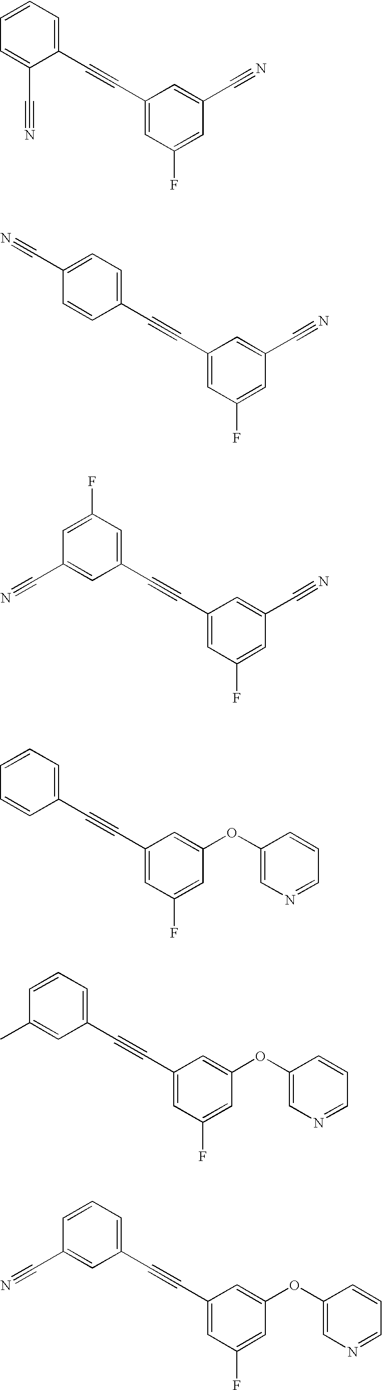 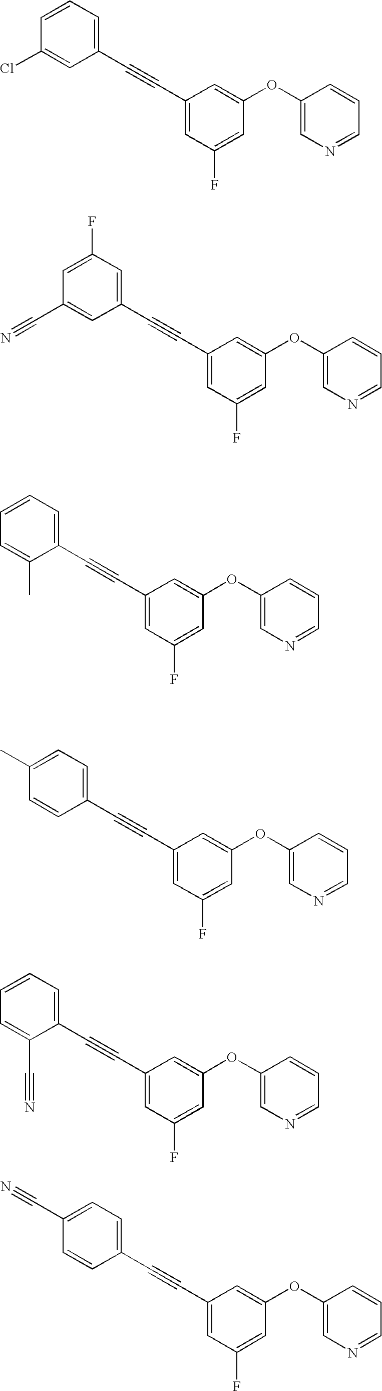 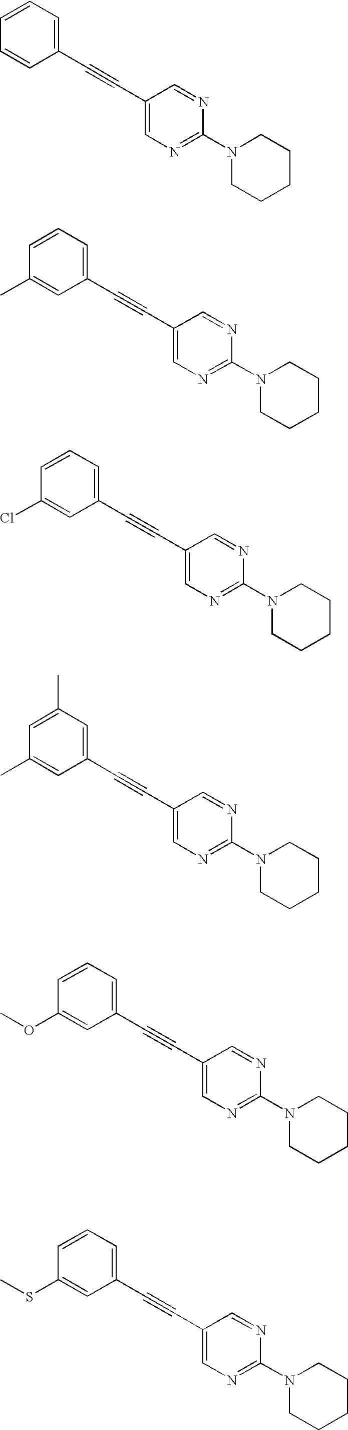 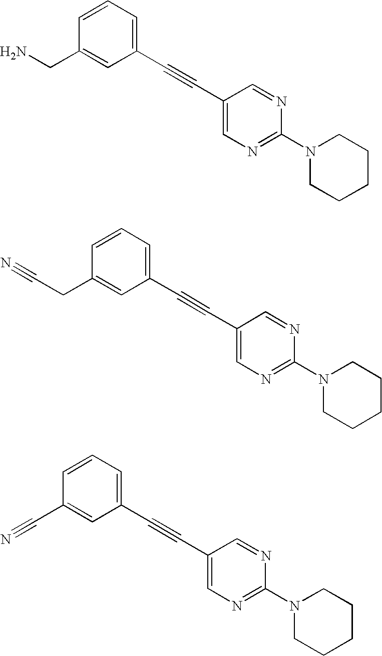
or a pharmaceutically acceptable salt thereof.

20. A pharmaceutical composition comprising a pharmaceutically acceptable carrier and the compound of claim 8 or a pharmaceutically acceptable salt thereof.
